(19) United States
(12) Patent Application Publication
(10) Pub. No.: US 2006/0188461 A1
(43) Pub. Date: Aug. 24, 2006

(54) Linear sulfonic polyester as a water-resistant hair fixing agent

(76) Inventors: Cecile Bebot, Clichy (FR); Ludivine Laurent, Courbevoie (FR); Sabine Vrignaud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/177,479

(22) Filed: Jul. 11, 2005

Related U.S. Application Data
(60) Provisional application No. 60/591,860, filed on Jul. 29, 2004.

(30) Foreign Application Priority Data
Jul. 29, 2004 (FR) 04 51706

(51) Int. Cl. A61K 8/85

(52) U.S. Cl. 424/70.11

(57) ABSTRACT

The present patent application relates to the use of at least one water-dispersible linear sulfonic polyester as a hair fixing agent that is resistant to liquid water.

The present patent application relates to the use of at least one water-dispersible linear sulfonic polyester as a water-resistant hair fixing agent.

Cosmetic compositions for shaping and/or holding the hairstyle may be spray compositions consisting essentially of a solution, which is usually alcoholic, and of one or more components known as fixing components, which are generally polymer resins, the function of which is to form welds between the hairs or to coat the hairs. These fixing components are often formulated as a mixture with various cosmetic adjuvants.

These cosmetic compositions are generally packaged either in a pump-disperser bottle or in a suitable aerosol container pressurized using a propellant, and the aerosol system then contains firstly a liquid phase (or fluid) and secondly a propellant.

Styling compositions in the form of gels, creams or mousses may also be used.

Once applied to the hair, the fixing compounds should allow fixing of the hair.

However, the polymers commonly used as fixing agents in styling compositions do not allow the shape of the hairstyle to be conserved when the hair is placed in contact with liquid water for a prolonged period, for instance contact with rain, perspiration or during bathing: for example bathing in the sea or in a swimming pool.

The Applicant has discovered, surprisingly and advantageously, that the use of at least one water-dispersible linear sulfonic polyester, in a cosmetically acceptable medium, at a concentration of between 5% and 40% by weight relative to the total weight of the cosmetic composition, has the effect of fixing and shaping the hairstyle and also of conserving the shape of the hairstyle when the hair is placed in contact with liquid water for a prolonged period.

For the purposes of the present patent application, this phenomenon is referred as “resistance to liquid water”.

For the purposes of the present patent application, the term “prolonged period” means contact with water for a minimum period of one minute, preferably ten minutes and even more preferably 20 minutes.

The conservation of the shape may be evaluated by a metrological test. The test protocol is as follows.

Procedure:

Apply 2 g of the test formulation to a lock of 2.7 g of natural hair 27 cm long.
Roll up the treated lock on a curler 1 cm in diameter to give it a shape.
Leave the product to dry in air, and then delicately unroll the lock from the curler.
The locks thus shaped are then immersed in a bath of saline water (3% NaCl) of 8 litres volume at room temperature, with magnetic stirring at 100 rpm.
Measure the length of the lock over time in order to evaluate the shape hold.
   Measurements of the Shape Hold:
% shape hold: (L₁−L)/(L₁−L₀)*100
L: length of the curled lock at time t
L₀: length of the curled lock after shaping and removal from the curler
L₁: length of the lock before shaping on the curler

Preferably, compositions that allow a shape hold of greater than 70% for an immersion time of 10 minutes are selected: this is then referred to as water resistance. With the linear sulfonic polyesters of the invention, a shape conservation of this type is obtained.

The compositions of the invention also allow atmospheric moisture-resistant styling to be obtained.

The compositions according to the present invention allow good fixing and good hold of the hair, i.e. a styling effect that lasts throughout the day, or even for several days, which has good water resistance, especially good resistance to repeated bathing, these compositions also having the advantage of being removed by shampooing.

These compositions can also impart good cosmetic properties.

One subject of the present invention is the use, as a hair shape fixing agent that is resistant to liquid water, of at least one water-dispersible linear sulfonic polyester with a glass transition temperature of between 50 and 100° C., at a relative concentration of between 5% and 40% by weight, in a cosmetically acceptable medium.

The glass transition temperature (Tg) is measured by differential thermal analysis (DSC, differential scanning calorimetry) according to standard ASTM D 3418-97.

Advantageously, the cosmetically acceptable medium is free of fatty substances chosen from mineral oils, plant oils and waxes.

More advantageously, the cosmetically acceptable medium comprises between 20% and 95% by weight, preferably between 30% and 85% and more preferably between 40% and 80% by weight of water.

More advantageously, the cosmetically acceptable medium comprises between 0 and 20%, preferably between 0 and 10% and more preferably between 1% and 6% by weight of C1 to C4 alcohol.

The cosmetic composition used according to the invention may be in any form: lotions, sprays, mousses, gels, creams.

Preferably, the viscosity of the cosmetic composition is between 1 cps and 10⁵ cps, measured at 25° C. with a shear rate of 1 s⁻¹.

Other subjects, characteristics, aspects and advantages of the invention will emerge even more clearly on reading the description and the examples that follow.

For the purposes of the present patent application, the term “styling cosmetic composition” means a composition for shaping or holding the hairstyle.

The composition according to the invention comprises a water-dispersible linear sulfonic polyester.

The term “water-dispersible linear sulfonic polyester” means any sulfonic polyester capable of forming a dispersion, i.e. a two-phase system in which the first phase is formed from finely divided particles uniformly distributed in the second phase, which is the continuous phase.

The term “sulfonic polyester” means copolyesters obtained by polycondensation of at least one dicarboxylic acid or of an ester thereof, of at least one diol and of at least one sulfoaryldicarboxylic difunctional compound substituted on the aromatic nucleus with a group —SO₃M in which M represents a hydrogen atom or a metal ion such as Na⁺, Li⁺ or K⁺.

The water-dispersible linear sulfonic polyesters generally have a weight-average molecular mass of between about 1000 and 60 000 and preferably from 4000 to 20 000, as determined by gel permeation chromatography (or GPC).

The glass transition temperature (Tg) of these sulfonic polyesters is preferably within the range from 50° C. to 100° C. and preferably from 50° C. to 70° C.

They are described in greater detail in patent applications U.S. Pat. No. 3,734,874, U.S. Pat. No. 3,779,993, U.S. Pat. No. 4,119,680, U.S. Pat. No. 4,300,580, U.S. Pat. No. 4,973,656, U.S. Pat. No. 5,660,816, U.S. Pat. No. 5,662,893, and U.S. Pat. No. 5,674,479.

Advantageously, the water-dispersible linear sulfonic polyester is a polycondensate of at least one dicarboxylic acid or of an ester thereof, of at least one diol and of at least one sulfoaryldicarboxylic difunctional compound substituted on the aromatic nucleus with a group —SO₃M in which M represents a hydrogen atom or a metal ion such as Na⁺, Li⁺ or K⁺.

More advantageously, the water-dispersible linear sulfonic polyester is obtained from isophthalic acid, from the sodium salt of sulfoisophthalic acid, from diethylene glycol and from 1,4-cyclohexane-methanol.

The sulfonic polyesters preferably used in the invention comprise at least units derived from isophthalic acid, from a salt of sulfoaryldicarboxylic acid and from diethylene glycol, and more particularly, the sulfonic polyesters used in the invention are obtained from isophthalic acid, from the sodium salt of sulfoisophthalic acid, from diethylene glycol and from 1,4-cyclohexanemethanol.

Examples of sulfonic polyesters that may especially be mentioned include those known under the INCI name Diglycol/CHDM/Isophthalates/SIP and sold under the trade name “Eastman AQ polymer” (by the company Eastman Chemical).

The concentration of water-dispersible linear sulfonic polyester(s) used in the compositions according to the present invention is between 5% and 40%, preferably between 5% and 30% and even more preferably between 5% and 25% by weight relative to the total weight of the composition.

The styling composition according to the invention may also contain at least one additive chosen from nonionic, anionic, cationic and amphoteric surfactants, additional nonionic, anionic, cationic and amphoteric polymers, ceramides and pseudo ceramides, vitamins and provitamins including panthenol, water-soluble and liposoluble, silicone or non-silicone sunscreens, fillers and solid particles, for instance coloured or uncoloured mineral and organic pigments, nacreous agents and opacifiers, flakes, active particles, dyes, sequestering agents, plasticizers, solubilizers, acidifying agents, basifying agents, neutralizers, mineral and organic thickeners, antioxidants, hydroxy acids, penetrating agents, fragrances and preserving agents.

A person skilled in the art will take care to select the optional additives and the amount thereof such that they do not harm the properties of the compositions according to the present invention.

These additives are present in the composition according to the invention in an amount ranging from 0 to 20% by weight relative to the total weight of the composition.

The composition of the invention may be in the form of an aerosol or non-aerosol spray, a mousse, a gel, a paste or a cream.

Advantageously, the invention relates to the use of at least one linear sulfonic polyester with at least one nonionic surfactant for the formulation of a styling mousse, and also to the use of at least one linear sulfonic polyester to obtain compositions allowing a shape hold of greater than 70% for an immersion time of 10 minutes in a water resistance test.

The examples that follow illustrate the present invention and should not in any way be considered as limiting the invention.

### EXAMPLES

The following formulations are prepared:

Non-aerosol spray:

| | % active |
|---|---|
| Formulation A | material |
| Diglycol/CHDM/Isophthalates/SIP copolymer | 10 |
| (Eastman AQ 55S - Eastman) | |
| Deionized water | qs 100 |
| Fragrance and ingredients | qs |

Styling mousse

| | % active |
|---|---|
| Formulation B | material |
| Diglycol/CHDM/Isophthalates/SIP copolymer | 10 |
| (Eastman AQ 55S - Eastman) | |
| Oxyethylenated (20 OE) sorbitan monolaurate | 0.5 |
| Isobutane/propane (85/15) | 5 |
| Deionized water | qs 100 |
| Fragrance and ingredients | qs |

Aerosol spray

| | % active |
|---|---|
| Formulation C | material |
| Diglycol/CHDM/Isophthalates/SIP copolymer | 10 |
| (Eastman AQ 55S - Eastman) | |
| DME | 40 |
| Deionized water | qs 100 |
| Fragrance and ingredients | qs |

Measurement of the shape hold:

| Immersion time | | A | B | C |
|---|---|---|---|---|
| 0 | | 100 | 100 | 100 |
| 25 | seconds | 100 | 100 | 100 |
| 30 | seconds | 100 | 100 | 100 |
| 35 | seconds | 100 | 100 | 100 |
| 40 | seconds | 100 | 100 | 100 |
| 10 | minutes | 100 | 100 | 100 |

1. Use, as a hair shape fixing agent that is resistant to liquid water, of at least one water-dispersible linear sulfonic polyester with a glass transition temperature of between 50 and 100° C., at a relative concentration of between 5% and 40% by weight, in a cosmetically acceptable medium.

2-8. (canceled)
